(19) United States
(12) Patent Application Publication
(10) Pub. No.: US 2013/0225560 A1
(43) Pub. Date: Aug. 29, 2013

(54) Treatment of Cognitive Disorders with Certain Alpha-7 Nicotinic Acid Receptor Agonists in Combination with Acetylcholinesterase Inhibitors

(71) Applicants: Gerhard Koenig, Newton, MA (US); Dana Hilt, Waltham, MA (US)

(72) Inventors: Gerhard Koenig, Newton, MA (US); Dana Hilt, Waltham, MA (US)

(73) Assignee: ENVIVO PHARMACEUTICALS, INC., Watertown, MA (US)

(21) Appl. No.: 13/812,260

(22) PCT Filed: Jul. 25, 2011
(86) PCT No.: PCT/US2011/045206

§ 371 Date: May 8, 2013

Related U.S. Application Data
(60) Provisional application No. 61/367,672, filed on Jul. 26, 2010.
(60) Provisional application No. 61/411,911, filed on Nov. 9, 2010.
(60) Provisional application No. 61/412,353, filed on Nov. 10, 2010.

(51) Int. Cl. A61K 31/439; A61K 31/501; A61K 31/55; A61K 31/473; A61K 31/325; A61K 31/444; A61K 31/445

(52) U.S. Cl. CPC A61K 31/439; A61K 31/444; A61K 31/501; A61K 31/445; A61K 31/473; A61K 31/325; A61K 31/55
USPC 514/215; 514/305; 514/252.04; 514/297

(57) ABSTRACT

A method for improving cognition comprising administering to a patient certain alpha-7 receptor agonists and an acetylcholinesterase inhibitor is described together with related compositions.

### RELATED APPLICATIONS

This application claims benefit of U.S. Provisional Application No. 61/367,672, filed Jul. 26, 2010; U.S. Provisional Application No. 61/411,911, filed Nov. 9, 2010; and U.S. Provisional Application No. 61/412,353, filed Nov. 10, 2010, which are hereby expressly incorporated herein by reference in their entireties.

### BACKGROUND OF THE INVENTION

Nicotinic acetylcholine receptors (nAChR) form a family of ion channels activated by acetylcholine. Functional receptors contain five subunits and there are numerous receptor subtypes. Studies have shown that central nicotinic acetylcholine receptors are involved in learning and memory. Nicotinic acetylcholine receptors of the alpha-7 subtype (“alpha-7 receptors”) are prevalent in the hippocampus and cerebral cortex.

Some attribute the cognitive and functional decline observed in Alzheimer's patients to a cholinergic deficient in the central nervous system. At least four drugs that have been used to treat Alzheimer's Disease, tacrine, donepezil (donepezil HCl; 1-benyzl-4-[(5,6-dimethoxy-1-indanon)-2-yl]methylpiperidine monohydrochloride), rivastigmine ((S)—N-Ethyl-N-methyl-3-[1-(dimethylamino)ethyl]-phenyl carbamate) and galantamine (galantamine hydrobromide; (4aS,6R,8aS)-4a,5,9,10,11,12-hexahydro-3-methoxy-11-methyl-6H-benzofuro[3a,3,2-ef][2]benzazepin-6-ol hydrobromide), appear to act as acetylcholinesterase inhibitors that increase acetylcholine in the central nervous system.

N-[2-(pyridin-3-ylmethyl)-1-azabicyclo[2.2.2]oct-3-yl]-1-benzofuran-2-carboxamide (TC-5619) is reported to bind the alpha-7 receptor with very high affinity and to act as a full agonist of the receptor with little or no activity at other nicotinic receptors. In a murine model that exhibits many of the developmental, anatomical, and multi-transmitter biochemical aspects schizophrenia, TC-5619 acted both alone and in combination with clozapine to correct impaired pre-pulse inhibition (PPI) and social behavior which model positive and negative symptoms, respectively (Hauser et al. 2009 Biochemical Pharmacology 78:803).

### SUMMARY OF THE INVENTION

Described herein are methods for improving cognition in Alzheimer's patients by administering an acetylcholinesterase inhibitor (e.g., donepezil or rivastigmine) in combination with certain alpha-7 nicotinic acetylcholine receptor agonists (“alpha-7 agonists”). Useful alpha-7 receptor agonists include: N-[2-(pyridin-3-ylmethyl)-1-azabicyclo[2.2.2]oct-3-yl]-1-benzofuran-2-carboxamide (TC-5619, Formulae IVA-IVD), (S)—N-(quinuclidin-3-yl)-1H-indazole-3-carboxamide (Formula II), (R)-3-(6-(1H-indol-5-yl)pyridazin-3-yloxy)quinuclidine (Formula I), and (S)-1-(2-fluorophenyl)ethyl (S)-quinuclidin-3-ylcarbamate (Formula III) as well as enantiomers and pharmaceutically acceptable salts thereof. The methods can improve cognition in patients that have already benefited from an increase in one or more aspects of cognition due to the administration of an acetylcholinesterase inhibitor. Thus, a patient already benefiting from administration of an acetylcholinesterase inhibitor in one or more aspects of cognition can gain further benefit in one or more aspects of cognition from administration of an alpha-7 agonist described herein. Such alpha-7 agonists include

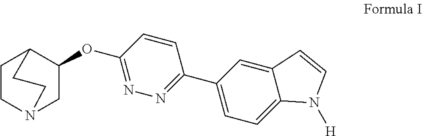 [SMILES: c1cc2cc(-c3ccc(O[C@H]4CN5CCC4CC5)nn3)ccc2[nH]1]

(R)-3-(6-(1H-indol-5-yl)pyridazin-3-yloxy)quinuclidine;

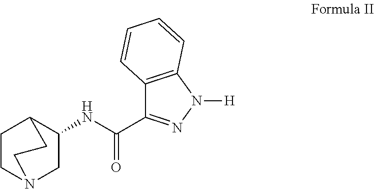 [SMILES: O=C(N[C@@H]1CN2CCC1CC2)c1n[nH]c2ccccc12]

(S)—N-(quinuclidin-3-yl)-1H-indazole-3-carboxamide or a mixture comprising (S)—N-(quinuclidin-3-yl)-1H-indazole-3-carboxamide and (R)—N-(quinuclidin-3-yl)-1H-indazole-3-carboxamide;

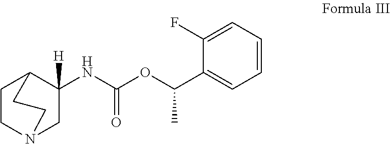 [SMILES: C[C@H](OC(=O)N[C@@H]1CN2CCC1CC2)c1ccccc1F]

(S)-1-(2-fluorophenyl)ethyl (S)-quinuclidin-3-ylcarbamate;

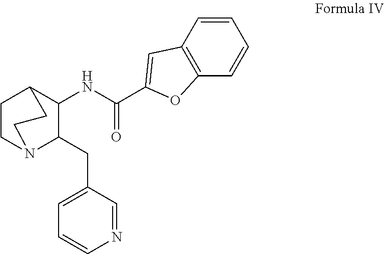 [SMILES: O=C(NC1C2CCN(CC2)C1Cc1cccnc1)c1cc2ccccc2o1]

N-[2-(pyridin-3-ylmethyl)-1-azabicyclo[2.2.2]oct-3-yl]-1-benzofuran-2-carboxamide, wherein the enantiomeric pairs that comprise the four diastereomers described by this structural formula are shown below as Formulae IVA-IVB, each of which is intended to be covered within the scope of the present invention:

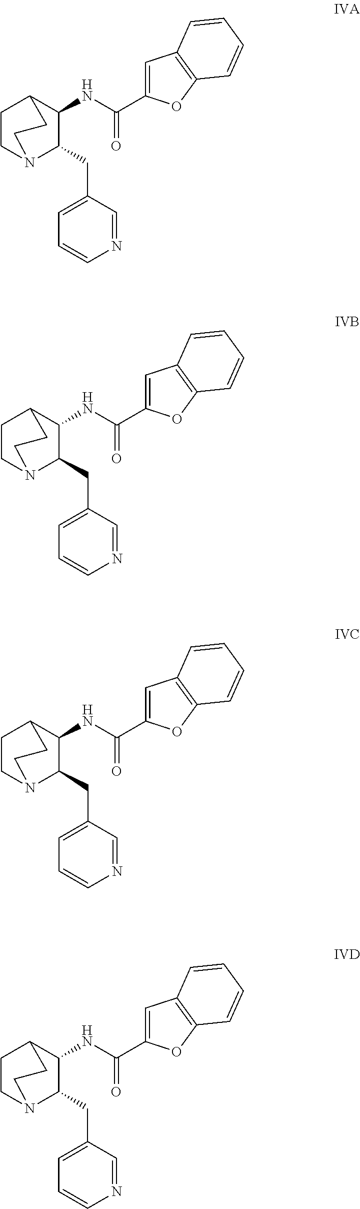 [SMILES: O=C(N[C@@H]1C2CCN(CC2)[C@H]1Cc1cccnc1)c1cc2ccccc2o1 ; O=C(N[C@H]1C2CCN(CC2)[C@@H]1Cc1cccnc1)c1cc2ccccc2o1 ; O=C(N[C@@H]1C2CCN(CC2)[C@@H]1Cc1cccnc1)c1cc2ccccc2o1 ; O=C(N[C@H]1C2CCN(CC2)[C@H]1Cc1cccnc1)c1cc2ccccc2o1]

or (2S, 3R)-N-[2-(pyridin-3-ylmethyl)-1-azabicyclo[2.2.2]oct-3-yl]-1-benzofuran-2-carboxamide, (2R, 3S)-N-[2-(pyridin-3-ylmethyl)-1-azabicyclo[2.2.2]oct-3-yl]-1-benzofuran-2-carboxamide, (2S, 3S)-N-[2-(pyridin-3-ylmethyl)-1-azabicyclo[2.2.2]oct-3-yl]-1-benzofuran-2-carboxamide, and (2R, 3R)-N-[2-(pyridin-3-ylmethyl)-1-azabicyclo[2.2.2]oct-3-yl]-1-benzofuran-2-carboxamide.

In certain embodiments of the invention, the alpha-7 receptor agonist is selected from one of the compounds of Formula I, II, III, IVA, IVB, IVC, IVD, pharmaceutically acceptable salts thereof, polymorphs thereof, hydrates thereof, solvates thereof, monohydrochlorides thereof, or solvates or hydrates of monohydrochlorides thereof. In a particular embodiment, the alpha-7 receptor agonist is (2S, 3R)-N-[2-(pyridin-3-ylmethyl)-1-azabicyclo[2.2.2]oct-3-yl]-1-benzofuran-2-carboxamide or a pharmaceutically acceptable salt or polymorph thereof.

Compounds in the disclosure may be in the form of pharmaceutically acceptable salts. The phrase “pharmaceutically acceptable” is a well-known term of art that refers to salts prepared from pharmaceutically acceptable non-toxic bases and acids, including inorganic and organic bases and inorganic and organic acids. Salts derived from inorganic bases include lithium, sodium, potassium, magnesium, calcium and zinc. Salts derived from organic bases include ammonia, primary (e.g. Tromethamine), secondary and tertiary amines, and amino acids (e.g. Lysine). Salts derived from inorganic acids include sulfuric, hydrochloric, phosphoric, methanesulphonic, hydrobromic. Salts derived from organic acids include C₁₋₆ alkyl carboxylic acids, di-carboxylic acids and tricarboxylic acids such as acetic acid, propionic acid, fumaric acid, maleic acid, succinic acid, tartaric acid, adipic acid and citric acid, and alkylsulfonic acids such as methanesulphonic, and aryl sulfonic acids such as para-tolouene sulfonic acid and benzene sulfonic acid. For detailed list of slats see P. H. Stahl and C. G. Wermuth (eds.) “Handbook of Pharmaceutical Salts, Properties, Selection and Use” Wiley-VCH (ISBN 3-906390-26-8)

Compounds and pharmaceutically acceptable salts thereof may be in the form of a solvate. This occurs when a compound of the invention forms a complex (e.g., crystallizes) with one or more solvent molecules. Examples of solvents forming solvates are water (hydrates), MeOH, EtOH, iPrOH, and acetone. Compounds of the invention described herein cover all solvates of the depicted compounds.

Compounds in the disclosure may exist in different crystal forms known as polymorphs.

In certain cases one or both of the acetylcholinesterase inhibitor and the alpha-7 agonist can be administered at a dosage which is sub-clinical in the absence of the other compound, i.e., at a dosage at which the compound (administered in the absence of the other compound) would not have a clinically measurable benefit on cognition (e.g., at a dose that does not measurably improve memory). Thus, a patient can experience a benefit (e.g., improved memory or cognition) from a combination of drugs each of which is administered at very low, side-effect reducing or side-effect avoiding dose. Moreover, the combination of drugs may provide a benefit for a wider range or patients and/or over a longer period of treatment. For example, while certain acetylcholinesterase inhibitors can exhibit reduce efficacy after several months of treatment, the combination may provide a longer period of efficacy.

A patient can benefit in one or more of: speed of processing, attention/vigilance, working memory, visual learning, verbal learning, visual learning, reasoning/problem solving, executive function, and social cognition. Importantly, the alpha-7 agonists can be used at low doses in such already treated patients to improve cognition, for example at a daily oral dose of (or no more than): 3 mg, 2.70 mg, 2.50 mg, 2.25 mg, 2 mg, 1.75 mg, 1.50 mg, 1.25 mg, 1 mg, 0.7, 0.5, 0.3 mg or even 0.1 mg. Thus, for example, they can be administered at 0.05-1.5 mg daily dose, e.g., 1 mg/daily or 0.5 mg/daily. For donepezil, the daily dose used with an alpha-7 agonist can be 10 mg, 5 mg, 4.5 mg, 4 mg, 3.5 mg, 3 mg, 2.5 mg, 2 mg, 1 mg or 0.5 mg. The daily dose can be between 5 and 0.5 mg (e.g., 4.5-1.0 mg/day, 4.5-2.0 mg/day, 4.0-2.0 or 2.5 mg/day). For rivastigmine the daily dose for use in combination can be 11, 10, 9, 8, 7, 6 or 5 mg. For galantamine the daily dose for use in combination can be 20, 15, 13, 12, 11, 10, 9, 8, 7, 6 or 5 mg. For acetylcholinesterase inhibitors it is understood that for effectiveness in improving memory or cognition, they must be administered so as to achieve a red blood cell acetylcholinesterase inhibition of at least 65%. In the methods described herein the acetylcholinesterase inhibitor can be administered at a lower dose that achieves only 60%, 55% (50, 45, 40, 35 or 30% inhibition) in the absence of the alpha-7 agonist.

Described herein is a method for improving cognition, the method comprising administering to a patient an alpha-7 agonist and an acetylcholinesterase inhibitor wherein one or both compounds are administered at a sub-clinical dose (i.e., a dose that would be subclinical in the absence of the other compound. In various cases: the patient has been diagnosed with Alzheimer's disease or pre-Alzheimer's disease, the patient has been diagnosed with mild to moderate Alzheimer's disease, the patient has been diagnosed with moderate to severe Alzheimer's disease, the acetylcholinesterase inhibitor is selected from tacrine, donepezil, rivastigmine and galantamine, the acetylcholinesterase inhibitor is selected from donepezil, rivastigmine and galantamine, the acetylcholinesterase inhibitor is selected from donepezil and rivastigmine, the patient has been administered an acetylcholinesterase inhibitor for a period of time prior to being administered an alpha-7 agonist, the patient has been administered an alpha-7 agonist for a period of time prior to being administered an acetylcholinesterase inhibitor, the prior administration has been for at least one month, the prior administration has been for at least three months, and the prior administration has been for at least six months. In certain cases: the method improves one or more of: learning, delayed memory, attention, working memory, visual learning, speed of processing, vigilance, verbal learning, visual motor function, social cognition, long term memory or executive function.

Also described is a method for improving cognition comprising administering to a patient an alpha-7 agonist at a subclinical dose (a dose that does not improve memory when administered in the absence of an acetylcholinesterase inhibitor) and an acetylcholinesterase inhibitor, also at a subclinical dose (a dose that does not improve memory when administered in the absence of the alpha-7 agonist. In various cases: the alpha-7 agonist is orally administered at 3.0 mg/day, 1.0 mg/day; 0.5 mg/day; 0.3 mg/day; or 0.1 mg/day. In various cases: the acetylcholinesterase inhibitor is donepezil and is orally administered at 5 mg/day; 4.5 mg/day; 4.0 mg/day; 2.5 mg/day; 1.5 mg/day or less; 1.0 mg/day; and the acetylcholinesterase inhibitor is administered at a dose that achieves 10-65% steady state red blood cell acetylcholinesterase inhibition.

Also described is a pharmaceutical composition that comprises an acetylcholinesterase inhibitor and an alpha-7 agonist, for example, N-[2-(pyridin-3-ylmethyl)-1-azabicyclo[2.2.2]oct-3-yl]-1-benzofuran-2-carboxamide (TC-5619) (e.g., (2S, 3R)-N-[2-(pyridin-3-ylmethyl)-1-azabicyclo[2.2.2]oct-3-yl]-1-benzofuran-2-carboxamide, (2R, 3S)-N-[2-(pyridin-3-ylmethyl)-1-azabicyclo[2.2.2]oct-3-yl]-1-benzofuran-2-carboxamide, (2S, 3S)-N-[2-(pyridin-3-ylmethyl)-1-azabicyclo[2.2.2]oct-3-yl]-1-benzofuran-2-carboxamide, and (2R, 3R)-N-[2-(pyridin-3-ylmethyl)-1-azabicyclo[2.2.2]oct-3-yl]-1-benzofuran-2-carboxamide), (S)-1-(2-fluorophenyl)ethyl (S)-quinuclidin-3-ylcarbamate, (S)—N-(quinuclidin-3-yl)-1H-indazole-3-carboxamide, and (R)-3-(6-(1H-indol-5-yl)pyridazin-3-yloxy)quinuclidine), and a pharmaceutically acceptable carrier.

The alpha-7 agonists can also be selected from those described in any of the following patent applications and patents: WO 2004/029050 WO 2006/065233, US 2005/0245531, WO 2005/092890, WO 2007/038367, WO 2005/092890, WO 2002/7038367, U.S. Pat. No. 6,780,861 and U.S. Pat. No. 6,953,855.

In various cases: the acetylcholinesterase inhibitor is selected from tacrine, donepezil, rivastigmine and galantamine; the acetylcholinesterase inhibitor is selected from donepezil, rivastigmine and galantamine; the acetylcholinesterase inhibitor is selected from donepezil and rivastigmine; and the acetylcholinesterase inhibitor is donepezil.

Also described is a daily unit dosage pharmaceutical composition that comprises no more than 3.0 mg, 1.0 mg of 0.5 mg of an alpha-7 agonist, an acetylcholinesterase inhibitor and a pharmaceutically acceptable carrier. In various cases the daily unit dosage pharmaceutical composition comprises no more than 1.0, 0.5 (0.3, or 0.1) mg of the alpha-7 agonist. In various cases the daily unit dosage pharmaceutical composition comprises no more than 5, 4, 3, 2, 1, or 0.5 mg of donepezil.

Also described is a packaged pharmaceutical combination comprising a package containing a first unit dosage of an alpha-7 agonist and a second unit dosage pharmaceutical composition comprising an acetylcholinesterase inhibitor.

Described herein are methods for treating a patient by administering a pharmaceutical composition that comprises an alpha-7 agonist (e.g., at a daily dose of: 3 mg, 2.70 mg, 2.50 mg, 2.25 mg, 2 mg, 1.75 mg, 1.50 mg, 1.25 mg, 1 mg, 0.7 mg, 0.5 mg, 0.3 mg, 0.1 mg, 0.03 mg or 0.01 mg) in combination with one or more inhibitors of acetylcholinesterase. The treatment can improve one or more facets of cognition (e.g., visual motor skill, learning, delayed memory, attention, working memory, visual learning, speed of processing, vigilance, verbal learning, visual motor function, social cognition, long term memory, executive function, etc.). The patient can have been treated with an acetylcholinesterase inhibitor for a period of time prior to the administration of the alpha-7 agonist or the patient can have been treated with the alpha-7 agonist for a period of time prior to administration of the acetylcholinesterase inhibitor. For example, the patient can have been treated with one or the other of the drugs for at least one week, at least one month, at least two months, at least three months, at least four months, at least 6 months or at least one year. The two agents can be administered at the same time either in the same composition or in two different compositions. In addition, the agents can be administered at different times.

Also described herein is a pharmaceutical composition that comprises an alpha-7 agonist and a acetylcholinesterase inhibitor (e.g., tacrine, donepezil, rivastigmine or galantamine) and a pharmaceutically acceptable carrier.

For oral administration, a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; in particular embodiments, such materials also include lactose or milk sugar as well as high molecular weight polyethylene glycols. When aqueous suspensions and/or elixirs are desired for oral administration, the compounds/components of this invention can be combined with various sweetening agents, flavoring agents, coloring agents, emulsifying agents and/or suspending agents, as well as such diluents as water, ethanol, propylene glycol, glycerin and various like combinations thereof. For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, may be prepared.

Methods of preparing various pharmaceutical compositions with a certain amount of active ingredient are known, or will be apparent in light of this disclosure, to those of ordinary skill in this art. For examples of methods of preparing pharmaceutical compositions, see Remington's Pharmaceutical Sciences, Mack Publishing Company, Easter, Pa., 15th Edition (1975). After a pharmaceutical composition has been formulated in an acceptable carrier, it can be placed in an appropriate container and labeled for treatment of an indicated condition. For administration of the compounds of the invention, such labeling would include, e.g., instructions concerning the amount, frequency and method of administration.

“Dose” is the amount of active pharmaceutical ingredient (API) administered to a patient. For example 1 mg means 1 mg of API was orally administered to each patient each day.

“Active Pharmaceutical Ingredient” includes the alpha-7 agonists and acetylcholinesterase inhibitors described herein.

### DETAILED DESCRIPTION OF THE INVENTION

The effects of the drug combinations described herein on cognition in human patients can be measured using CogState computerized cognitive testing and/or by using all or a subset of NTB scales (e.g., category fluency, Trails A and B).

The effects of the drug combinations described herein on short-term memory can be measured in a murine model by examining the effects on deficits induced by the muscarinic antagonist scopolamine in the object recognition task in 5-months-old male Wistar rats. The object recognition task allows the assessment of consolidation of (object) information into memory (Ennaceur and Delacour, 1988; Prickaerts et al., 1997). In this task a rat is given two trials. In the first trial the rat is put into an arena in which two identical objects are placed. Usually, a rat will inspect the two objects for a certain amount of time. After a certain delay, the rat is given a second trial. In this trial the rat is again placed in the same arena but one of the objects is replaced with a novel object. Similar to the first trial the rat again explores the two objects. The amount of time exploring each object is recorded in order to determine whether the rat spends a different amount of time exploring the two objects. On basis of this scoring the memory performance can be determined.

Several studies have shown that Wistar rats show a good object memory performance when a one-hour delay is interposed between the first and second trial. However, when a twenty-four hour delay is used the rats do not discriminate between the novel and the familiar object in the second trial, indicating that the rats do not remember the familiar object (i.e., the object presented in both the first trial and the second trial). Using a six-hour delay, the discrimination performance is between the performance of the one hour and twenty-four hour delay, suggesting a delay-dependent forgetting in this task.

Briefly, 30 minutes before the learning trial, rats receive an injection with the muscarinic receptor antagonist scopolamine (0.1 mg/kg, administered i.p.). After treatment with scopolamine, rats will show no memory of the objects one hour after the learning trial. Drugs to be tested are given 30 minutes before the first trial, i.e., just after administration of scopolamine.

The animals are individually housed in standard type 3 Makrolon cages on sawdust bedding in an air-conditioned room (about 20° C.). They are kept under a 12/12-hour light/dark cycle (lights on from 19.00 to 7.00 h) and have free access to food and water. Rats are housed in the same room where they are tested.

The object recognition test is performed as described elsewhere (Ennaceur and Delacour, 1988). The apparatus consists of a circular arena, 83 cm in diameter. Half of the 40 cm high wall is made of gray polyvinyl chloride, the other half of transparent polyvinyl chloride. The light intensity is equal in the different parts of the apparatus. Two objects are placed in a symmetrical position about 10 cm away from the gray wall. Each object is available in triplicate. The objects are: 1) a cone consisting of a gray polyvinyl chloride base (maximal diameter 18 cm) with a collar on top made of brass (total height 16 cm), 2) a standard 1 liter brown transparent glass bottle (diameter 10 cm, height 22 cm) filled with water, 3) a massive metal cube (10.0×5.0×7.5 cm) with two holes (diameter 1.9 cm), and 4) a massive aluminum cube with a tapering top (13.0×8.0×8.0 cm). A rat cannot displace the objects. Fluorescent red tubes and a light bulb provided a constant illumination of about 20 lux on the floor of the apparatus.

A testing session includes two trials. The duration of each trial is 3 min. During the first trial (T1) the apparatus contains two identical objects (samples). A rat is always placed in the apparatus facing the wall at the middle of the front (transparent) segment. After the first exploration period the rat is put back in its home cage. Subsequently, after a predetermined delay interval, the rat is put back in the apparatus for the second trial (T2), but now with two dissimilar objects, a familiar one (the sample) and a new one. The times spent in exploring each object during T1 and T2 are recorded manually with a personal computer.

Exploration is defined as follows: directing the nose to the object at a distance of no more than 2 cm and/or touching the object with the nose. Sitting on the object is not considered as exploratory behavior. In order to avoid the presence of olfactory trails the objects are always thoroughly cleaned. All combinations and locations of objects are used in a balanced manner to reduce potential biases due to preferences for particular locations or objects.

Several studies have shown that Wistar rats show a good object memory performance when a one-hour delay interposed between the first trial and the second trial. However, when a twenty-four hour delay is used the rats do not discriminate the novel and the familiar in the second trial, indicating that the rats do not remember the object that was presented in the first trial. Using a six hour delay, the discrimination performance is between the performance of the one hour and twenty-four hour delay, suggesting a delay-dependent forgetting in this task. In this study a 1 h interval is used.

In the two weeks prior to testing, the animals are handled daily and adapted to the procedure in two days, i.e., they are allowed to explore the apparatus (without any objects) twice for 3 min each day. Then the rats are adapted to the testing and i.p. and p.o. injections by a saline injection (1.0 ml/kg and 2.0 ml/kg respectively) 30 min before the first trial until they show a stable discrimination performance, i.e. a good discrimination at 1-h interval and no discrimination at 24-h interval.

The basic measures are the times spent by rats in exploring an object during T1 and T2. The time spent in exploring the two identical samples are represented by ‘a1’ and ‘a2’. The time spent in T2 in exploring the sample and new object are represented by ‘a’ and ‘b’, respectively. The following variables are calculated: e1=a1+a2, e2=a+b, and d2=(b−a)/e2. e1 and e2 are measures of the total exploration time of both objects during T1 and T2 respectively. D2 is a relative measure of discrimination corrected for exploration activity (e2). Thus, there should be no differences in d2 indices between experiments with similar treatments at similar intervals.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

1. A method for improving cognition comprising administering to a patient a compound selected from the group consisting of: (S)-1-(2-fluorophenyl)ethyl (S)-quinuclidin-3-ylcarbamate, N-[2-(pyridin-3-ylmethyl)-1-azabicyclo[2.2.2]oct-3-yl]-1-benzofuran-2-carboxamide (TC-5619), (S)—N-(quinuclidin-3-yl)-1H-indazole-3-carboxamide, and (R)-3-(6-(1H-indol-5-yl)pyridazin-3-yloxy)quinuclidine or a pharmaceutically acceptable salt thereof, and an acetylcholinesterase inhibitor.

2. The method of claim 1 wherein the patient has been diagnosed with Alzheimer's disease or pre-Alzheimer's disease.

3. The method of claim 1 wherein the patient has been diagnosed with mild to moderate Alzheimer's disease.

4. The method of claim 1 wherein the patient has been diagnosed with moderate to severe Alzheimer's disease.

5. The method of claim 1, wherein the acetylcholinesterase inhibitor is selected from tacrine, donepezil, rivastigmine and galantamine.

6. The method of claim 5 wherein the acetylcholinesterase inhibitor is selected from donepezil, rivastigmine and galantamine.

7. The method of claim 5 wherein the acetylcholinesterase inhibitor is selected from donepezil and rivastigmine.

8. The method of claim 1, wherein the patient has been administered an acetylcholinesterase inhibitor for a period of time prior to being administered a compound selected from the group consisting of: (S)-1-(2-fluorophenyl)ethyl (S)-quinuclidin-3-ylcarbamate, (2S, 3R)-N-[2-(pyridin-3-ylmethyl)-1-azabicyclo[2.2.2]oct-3-yl]-1-benzofuran-2-carboxamide, (S)—N-(quinuclidin-3-yl)-1H-indazole-3-carboxamide, and (R)-3-(6-(1H-indol-5-yl)pyridazin-3-yloxy)quinuclidine or a pharmaceutically acceptable salt thereof.

9. The method of claim 8 wherein the prior administration has been for at least one month.

10. The method of claim 9 wherein the prior administration has been for at least three months.

11. The method of claim 10 wherein the prior administration has been for at least six months.

12. The method of claim 1, wherein the method improves one or more of: learning, delayed memory, attention, working memory, visual learning, speed of processing, vigilance, verbal learning, visual motor function, social cognition, long term memory or executive function.

13. The method of claim 1 wherein one or both of the alpha-7 agonist and the acetylcholinesterase inhibitor is administered at a subclinical dose.

14. The method of claim 13 wherein the alpha-7 receptor agonist is orally administered at less than 1.0 mg/day.

15. The method of claim 13 wherein the alpha-7 receptor agonist is orally administered at less than 0.5 mg/day.

16. The method of claim 13 wherein the alpha-7 receptor agonist is orally administered at less than 0.3 mg/day.

17. The method of claim 13 wherein the alpha-7 receptor agonist is orally administered at less than 0.1 mg/day.

18. The method of claim 13 wherein the acetylcholinesterase inhibitor is donepezil and is orally administered at less than 5 mg/day.

19. The method of claim 13 wherein the acetylcholinesterase inhibitor is donepezil and is orally administered 4.5 mg/day or less.

20. The method of claim 13 wherein the acetylcholinesterase inhibitor is donepezil and is orally administered at 4.0 mg/day or less.

21. The method of claim 13 wherein the acetylcholinesterase inhibitor is donepezil and is orally administered at 2.5 mg/day or less.

22. The method of claim 13 wherein the acetylcholinesterase inhibitor is donepezil and is orally administered at 1.5 mg/day or less.

23. The method of claim 13 wherein the acetylcholinesterase inhibitor is donepezil and is orally administered at than 1.0 mg/day or less.

24. The method of claim 1 wherein the acetylcholinesterase inhibitor is administered at a dose that achieves 10-65% steady state red blood cell acetylcholinesterase inhibition.

25. A pharmaceutical composition comprising a compound selected from the group consisting of: (S)-1-(2-fluorophenyl)ethyl (S)-quinuclidin-3-ylcarbamate, N-[2-(pyridin-3-ylmethyl)-1-azabicyclo[2.2.2]oct-3-yl]-1-benzofuran-2-carboxamide (TC-5619), (S)—N-(quinuclidin-3-yl)-1H-indazole-3-carboxamide, and (R)-3-(6-(1H-indol-5-yl)pyridazin-3-yloxy)quinuclidine or a pharmaceutically acceptable salt thereof, an acetylcholinesterase inhibitor, and a pharmaceutically acceptable carrier.

26. The pharmaceutical composition of claim 25 wherein the acetylcholinesterase inhibitor is selected from tacrine, donepezil, rivastigmine and galantamine.

27. The pharmaceutical composition of claim 25 wherein the acetylcholinesterase inhibitor is selected from donepezil, rivastigmine and galantamine.

28. The pharmaceutical composition of claim 25 wherein the acetylcholinesterase inhibitor is selected from donepezil and rivastigmine.

29. The pharmaceutical composition of claim 25 wherein the acetylcholinesterase inhibitor is donepezil.

30. A daily unit dosage pharmaceutical composition comprising no more than 1.0 mg of a compound selected from the group consisting of: (S)-1-(2-fluorophenyl)ethyl (S)-quinuclidin-3-ylcarbamate, N-[2-(pyridin-3-ylmethyl)-1-azabicyclo[2.2.2]oct-3-yl]-1-benzofuran-2-carboxamide (TC-5619), (S)—N-(quinuclidin-3-yl)-1H-indazole-3-carboxamide, and (R)-3-(6-(1H-indol-5-yl)pyridazin-3-yloxy)quinuclidine or a pharmaceutically acceptable salt thereof, an acetylcholinesterase inhibitor and a pharmaceutically acceptable carrier.

31. The daily unit dosage pharmaceutical composition of claim 30 comprising no more than 0.5 mg of (a compound selected from the group consisting of: (S)-1-(2-fluorophenyl)ethyl (S)-quinuclidin-3-ylcarbamate, N-[2-(pyridin-3-ylmethyl)-1-azabicyclo[2.2.2]oct-3-yl]-1-benzofuran-2-carboxamide (TC-5619), (S)—N-(quinuclidin-3-yl)-1H-indazole-3-carboxamide, and (R)-3-(6-(1H-indol-5-yl)pyridazin-3-yloxy)quinuclidine or a pharmaceutically acceptable salt thereof.

32. The daily unit dosage pharmaceutical composition of claim 31 comprising no more than 0.3 mg a compound selected from the group consisting of: (S)-1-(2-fluorophenyl)ethyl (S)-quinuclidin-3-ylcarbamate, N-[2-(pyridin-3-ylmethyl)-1-azabicyclo[2.2.2]oct-3-yl]-1-benzofuran-2-carboxamide (TC-5619), (S)—N-(quinuclidin-3-yl)-1H-indazole-3-carboxamide, and (R)-3-(6-(1H-indol-5-yl)pyridazin-3-yloxy)quinuclidine or a pharmaceutically acceptable salt thereof.

33. The daily unit dosage pharmaceutical composition of claim 31 comprising no more than 0.1 mg a compound selected from the group consisting of: (S)-1-(2-fluorophenyl)ethyl (S)-quinuclidin-3-ylcarbamate, N-[2-(pyridin-3-ylmethyl)-1-azabicyclo[2.2.2]oct-3-yl]-1-benzofuran-2-carboxamide (TC-5619), (S)—N-(quinuclidin-3-yl)-1H-indazole-3-carboxamide, and (R)-3-(6-(1H-indol-5-yl)pyridazin-3-yloxy)quinuclidine or a pharmaceutically acceptable salt thereof.

34. The daily unit dosage pharmaceutical composition of claim 31 comprising no more than 5 mg of donepezil.

35. The daily unit dosage pharmaceutical composition of claim 31 comprising no more than 4 mg of donepezil.

36. The daily unit dosage pharmaceutical composition of claim 31 comprising no more than 2.5 mg of donepezil.

37. The daily unit dosage pharmaceutical composition of claim 31 comprising no more than 1 mg of donepezil.

38. Packaged pharmaceuticals comprising a package containing a first unit dosage pharmaceutical composition comprising a compound selected from the group consisting of: (S)-1-(2-fluorophenyl)ethyl (S)-quinuclidin-3-ylcarbamate, N-[2-(pyridin-3-ylmethyl)-1-azabicyclo[2.2.2]oct-3-yl]-1-benzofuran-2-carboxamide (TC-5619), (S)—N-(quinuclidin-3-yl)-1H-indazole-3-carboxamide, and (R)-3-(6-(1 H-indol-5-yl)pyridazin-3-yloxy)quinuclidine or a pharmaceutically acceptable salt thereof, and a second unit dosage pharmaceutical composition comprising an acetylcholinesterase inhibitor.
